(19) United States
(12) Patent Application Publication
(10) Pub. No.: US 2008/0177261 A1
(43) Pub. Date: Jul. 24, 2008

(54) INSTRUMENTATION FOR KNEE SURGERY

(76) Inventor: Derek James Wallace McMinn, Stourbridge (GB)

Correspondence Address: GOODWIN PROCTER LLP;PATENT ADMINISTRATOR, EXCHANGE PLACE, BOSTON, MA, 02109-2881, US

(21) Appl. No.: 11/972,830

(22) Filed: Jan. 11, 2008

(30) Foreign Application Priority Data
Jan. 13, 2007 (GB) 0700689.3

(51) Int. Cl. A61B 17/58

(52) U.S. Cl. 606/62; 606/90

(57) ABSTRACT

Instrumentation for use in knee surgery comprises an intramedullary rod (10) for insertion into an end of a femur (100) and a distraction device (62) coupleable to the intramedullary rod (10) and operable between the intramedullary rod (10) and the tibia for adjusting the tension of the collateral ligaments on either side of the knee. A cutting guide (70) is configured to locate over an intramedullary rod (10) that has been inserted into an end of a femur (100), such that the position of the cutting guide (70) relative to the intramedullary rod (10), and therefore the femur (100), is adjustable in at least the anterior-posterior direction. A kit for use in knee surgery is provided and a method of adjusting the tension of the collateral ligaments on either side of a knee is taught.

### FIELD OF THE INVENTION

This invention relates to instrumentation for use in knee surgery. Particularly, but not exclusively, the invention relates to instrumentation for use in total knee replacement (TKR) operations.

### BACKGROUND TO THE INVENTION

Prosthetic knee replacement surgery is employed to replace damaged or diseased parts of the knee. A particularly common disease that affects joints such as the knee is osteoarthritis. This disease usually increases in severity with age and leads to a high demand for successful knee replacement operations.

So-called total knee replacement (TKR) surgery involves replacing the ends of the femur and tibia with prosthetic femoral and tibial components. In some cases, the patella is also replaced with a patella component. During surgery, the patient's femur and tibia are resected, using appropriate cutting instruments, to the form required for mating with the respective femoral and tibial components.

The tibial component of a knee prosthesis typically comprises a plateau with a distal surface configured for mating with a resected proximal end of a tibia and a locating stem extending from the distal surface for insertion into the medullary canal of the tibia. Such a tibial component is usually cemented on to the proximal end of the tibia and a bearing component is provided on the proximal surface of the plateau to provide an articulating surface for cooperation with the femoral component.

The femoral component of a knee prosthesis is generally C-shaped with the external surface including medial and lateral condyles, which constitute articulating surfaces. The internal surface of the femoral component is usually constituted by a series of five planar surfaces angled successively. Thus, the resecting of the patient's femur must include the creation of corresponding planar surfaces to mate with those of the femoral component. In order to obtain such planar surfaces, a cutting guide is placed adjacent the distal end of the femur and cutting blades are inserted through appropriately angled slots in the guide.

Traditional instruments and techniques rely upon the surgeon's judgment as to whether the cutting guide is a) located in the correct anterior-posterior position with respect to the femur, and b) located in the correct rotational alignment with the femur. Mal-alignment of the cutting guide in either of these capacities will lead to incorrect location of the femoral component. This may lead to patient discomfort and poor performance of the joint. More specifically, if the cutting guide is located too far in the anterior direction, at least a portion of the anterior cuts will extend beyond the femur so that the resulting planar surface will be shorter than desired and the femoral component will be loose fitting. If the cutting guide is located too far in the posterior direction, the cuts will result in a divot or notch in the femur, which may lead to supracondylar fracture of the femur. Furthermore, if the cutting-guide, and therefore the femoral component, is rotationally mal-aligned, the collateral ligaments may not be under the correct tension.

During TKR surgery it important to obtain a flexion gap, i.e. the distance between the posterior femoral condyles and the proximal tibial surface when the knee is bent by 90°, that is equal to the extension gap, i.e. the distance between the distal femoral surface and the proximal tibial surface when the leg is straight. Usually, the extension gap is measured first and the flexion gap is then matched to the measured extension gap.

It is also important that the collateral ligaments and other soft tissue structures on either side of the knee are placed under the correct tension. Ideally, the lateral and medial collateral ligaments and soft tissue structures are placed under the same tension. If these ligaments and structures are not under equal tension the patient will have a feeling of instability and there is an increased risk that the bearing parts of the TKR will dislocate. A particular type of dislocation that may occur in mobile bearing designs is known as rotational spin out.

Conventionally, the desired tension of the lateral and medial collateral ligaments and soft tissue structures is achieved through the use of two independent distraction devices, each acting between one of the medial and lateral condyles and the proximal tibial surface. Such distraction devices are usually employed in spinal surgery and are large, heavy and cumbersome devices. Since they are operated independently there is a risk that the collateral ligaments and soft tissue structures on the medial and lateral sides of the knee will be unequally tensioned. Reliance is therefore placed on the surgeon to distract equally on both sides. In addition, these distraction devices require a significant amount of effort by the surgeon to operate since they generally involve lever-operated crank mechanisms. Furthermore, it is not uncommon for such devices to hinder access to the knee, during an operation. Moreover, these distraction devices tend to extend outside of the operating field of view so that their use is inconvenient.

An alternative distraction technique involves the use of L-shaped spacers that are successively inserted between the cutting guide and tibia to increase the distance therebetween. These only provide for step-wise rather than continual adjustment and they are awkward to use. In addition, to achieve an accurate tensioning with this technique it is necessary for the cutting guide to be held in fixed relationship with the femur. An inaccurate tensioning may therefore be provided if the cutting guide is not held in the correct fixed position.

A consequence of the above is that several large trays of instruments, typically 4-7, are required in TKR procedures. This results in the need to transport and autoclave a large number of devices before each operation.

Consequently, it is an aim of the present invention to address the shortcomings of the above.

### SUMMARY OF THE INVENTION

According to a first aspect of the present invention there is provided instrumentation for use in knee surgery comprising an intramedullary rod for insertion into an end of a femur and a distraction device coupleable to the intramedullary rod and operable between the intramedullary rod and a tibia for adjusting the tension of the collateral ligaments on either side of the knee.

Thus, the present invention enables a single distraction device to be employed to alter the tension of both the medial and lateral collateral ligaments. This reduces the number of instruments involved in the operation, provides for a simpler and quicker procedure, and increases the visibility and access to the knee during surgery. The present invention also allows for better control in setting and holding the desired tension during subsequent location and alignment of a cutting guide. As the distraction device operates between the intramedullary rod and the tibia there is no need for the device to contact fragile parts of the femur itself and so damage to the femur is minimised. Similarly, the distracting force may be applied to the tibia through a trial tibial component thereby preventing damage to the fragile surface of the tibia itself. In addition, the distraction procedure does not rely on the position of the cutting guide to obtain the desired tensioning. This results in the positioning of the cutting guide being independent of the distraction procedure, which leads to a greater degree of freedom when positioning the cutting guide. Embodiments of the present invention may allow for equal tensioning of the medial and lateral collateral ligaments and soft tissue structures on either side of the knee since the angle of the femur relative to the tibia is not fixed during the distraction procedure and so with the distraction device positioned centrally of the tibia the angle of the femur should naturally adjust to ensure the collateral ligaments are equally tensioned.

Note that for ease of reference throughout this specification, reference to a ‘tibia’ may include reference to a trial tibial component, a prosthetic tibial component or an actual tibia except where the context otherwise provides.

Optional but preferred features that may be adopted in embodiments of the invention are described below.

The distraction device may be securable to the intramedullary rod.

The distraction device may comprise two or more relatively displaceable members. In a specific embodiment, one of these members is configured to be coupled to the intramedullary rod and an other of these members is configured for displacement relative to the one member, in directions that generally correspond to the anterior and posterior directions of the femur when the instrumentation is in use. Optionally, the one member comprises an internal threaded portion in threaded engagement with a corresponding screw thread provided on the other member. The threaded engagement allows for fine control of the relative displacement of the two members. The one member may comprise an aperture through which the intramedullary rod may pass thereby to establish coupling engagement therebetween. In a particular embodiment, the one member is generally tubular having a longitudinal axis, and the aperture has an axis lying substantially orthogonal thereto and substantially co-incident with the longitudinal axis of the intramedullary rod, during the coupling engagement. The free end of the other member may be blunt or may taper to a tip. The other member may include a shoulder at or adjacent its free end for stabilising the orientation of the distraction device relative to the tibia.

The instrumentation may further comprise a device for coupling the distraction device to the tibia. Such a device may be configured to allow adjustment of the position of the distraction device relative to the tibia. Optionally, the device will permit adjustment in the anterior and posterior directions of the tibia. The device may be configured to hold the distraction device perpendicularly to the proximal end of the tibia. Furthermore, the device may be configured to permit flexion and/or extension of the knee during use. In a particular embodiment, the device may comprise a socket for receiving the free end of the distraction device, the socket being radially mounted on a wheel configured for rotation in a channel of a trial tibial component, the wheel being rotatably mounted on an axle configured for rolling along a plateau of a trial tibial component.

The instrumentation may further comprise a tool engageable with the other member and rotatable relative to the one member to facilitate extension and/or contraction of the distraction device. Optionally, the intramedullary rod includes an aperture along its length at the point of intersection of the longitudinal axis of the one member and the longitudinal axis of the intramedullary rod to allow said tool to access the other member. The tool may be configured as a torque screwdriver to enable to the surgeon to supply the correct collateral ligament tension. This is useful because although the present invention allows for equal tensioning of the medial and lateral collateral ligaments, the surgeon's feel is still required to judge the overall tensioning of the ligaments.

The instrumentation may further comprise a cutting guide positionable over the end of the femur such that the position of the cutting guide relative to the femur is adjustable. The cutting guide may be removably positionable over the end of the femur. Optionally, the cutting guide is configured to locate over the intramedullary rod when the instrumentation is in use. Additionally or alternately, the cutting guide is further configured to locate over the distraction device when the instrumentation is in use.

In a particular embodiment, the other member is accessible by the tool when the cutting guide is located over the intramedullary rod and the distraction device. It is particularly advantageous if the cutting guide is configured to locate over the distraction device and the intramedullary rod such as to be positionally adjustable, when the instrumentation is in use, in the anterior-posterior direction relative to the femur. The cutting guide may be coupleable to the distraction device and the intramedullary rod. Such coupling may be achieved by locating the cutting guide over the distraction device and the intramedullary rod through movement in one direction and then sliding the cutting guide relative to the distraction device and the intramedullary rod in an other direction. Optionally, the distraction device comprises an external surface profile that is slideably engageable with a corresponding surface provided in the cutting guide so as to provide said positional adjustability. The cutting guide may include a slot in which the intramedullary rod may slide so as to allow said positional adjustability. Furthermore, the cutting guide may include at least one stop to limit the amount of positional adjustability between the cutting guide and the intramedullary rod.

The instrumentation may further include a guide for providing an indication of the relative positional relationship between the cutting guide and the anterior of the femur. The guide may comprise a locating component for engagement with the cutting guide and an indicator spaced apart therefrom such that the indicator is disposed anteriorly of the femur when the instrumentation is in use, thereby to provide an indication of the relative positional relationship.

Moreover, the instrumentation may further include a rotation protractor configured to measure the degree of rotation between the cutting guide and the femur. Optionally, the rotation protractor comprises a reference portion configured to engage the cutting guide when in use and an indicator which extends beneath the lateral and medial condyles of the femur, the indicator being rotatable relative to the reference portion so as to contact the condyles thereby facilitating measurement of the relative angular relationship between the condyles and the cutting guide. The reference portion may be provided with a scale and the indicator may be provided with means for referencing the scale so as to provide the relative angular relationship.

According to a second aspect of the present invention there is provided instrumentation for use in knee surgery comprising a cutting guide configured to locate over an intramedullary rod which has been inserted into an end of a femur, such that the position of the cutting guide relative to the intramedullary rod, and therefore the femur, is adjustable in at least the anterior-posterior direction.

An advantage of the second aspect of the present invention is that the location of the cutting guide on the femur is not governed by the location of the intramedullary rod. This allows the surgeon a greater degree of freedom when positioning the cutting guide and therefore allows for the cutting guide to be more accurately located.

The instrumentation of the second aspect of the invention may further comprise a guide for providing an indication of the relative positional relationship between the cutting guide and the anterior and/or posterior of the femur.

Additionally or alternately, the instrumentation of the second aspect of the invention may further comprise a rotation protractor configured to measure the degree of rotation between the cutting guide and the femur.

According to a third aspect of the present invention there is provided a kit for use in knee surgery, the kit comprising two or more of the following instruments: an intramedullary rod for insertion into an end of a femur; a distraction device coupleable to an intramedullary rod and operable between the intramedullary rod and a tibia for adjusting the tension of the collateral ligaments on either side of the knee; a cutting guide configured to locate over an intramedullary rod which has been inserted into an end of a femur, such that the position of the cutting guide relative to the intramedullary rod, and therefore the femur, is adjustable in at least the anterior-posterior direction; a guide for providing an indication of the relative positional relationship between a cutting guide and the anterior and/or posterior of the femur; and a rotation protractor configured to measure the degree of rotation between a cutting guide and the femur.

The third aspect of the present invention therefore provides a kit that may selectively contain the instrumentation of the first and second aspects with their associated advantages as described above. In addition, the kit may include a guide to help determine the correct anterior/posterior position of the cutting guide with respect to the femur and a rotation protractor to help determine the correct angle of the cutting guide with respect to the femur.

According to a fourth aspect of the present invention there is provided a tibial component for use in knee surgery comprising: a plateau with a distal surface configured for mating with a proximal end of a resected tibia, a locating stem extending from the distal surface for insertion into a medullary canal of the tibia, and an annular protrusion extending from the distal surface to aid in attachment of the tibial component to the tibia, when in use.

An advantage of the fourth aspect of the present invention is that the annular protrusion provides for a secure attachment of the tibial component to a resected tibia. It can also be configured to act as a cement pressurising flange.

In one embodiment, the tibial component of the fourth aspect of the invention is configured as a trial tibial component and the protrusion is configured to create an annular cut in the proximal end of the tibia. The trial tibial component may include a recess in a proximal surface of the plateau for receiving a free end of a distraction device. Such a recess aids correct location of the distraction device with respect to the tibia and may be in the form of a hole or channel.

In another embodiment, the tibial component of the fourth aspect of the invention is configured as a prosthetic tibial component and the protrusion is configured to locate in an annular cut, such as that made by a trial tibial component as defined above, in the proximal end of the tibia. In a particular embodiment, the protrusion is configured as a cement pressurising flange to aid attachment of the prosthetic tibial component to the proximal end of the tibia by increasing the penetration of cement into the cancellous bone of the said proximal end of a tibia when in use.

According to a fifth aspect of the present invention there is provided a method of adjusting the tension of the collateral ligaments on either side of a knee, said method comprising the steps of: inserting an intramedullary rod into an end of a femur; and operating a distraction device between the intramedullary rod and a tibia to adjust the tension of the collateral ligaments on either side of the knee.

An advantage of the fifth aspect of the present invention is that only a single distraction device is required to alter the tension of both the medial and lateral collateral ligaments and so the procedure can be performed quickly and easily with increased visibility and access to the knee during the operation. Importantly, the present invention allows for equal tensioning of the medial and lateral collateral ligaments and soft tissue structures on either side of the knee since the angle of the femur relative to the tibia is not fixed during the distraction procedure and so with the distraction device positioned centrally of the tibia the angle of the femur should naturally adjust to ensure the collateral ligaments are equally tensioned. The present invention allows for better control in setting and holding the desired tension during subsequent steps of an operation, such as during the location and alignment of a cutting guide. As the distraction device is operated between the intramedullary rod and the tibia there is no need for the device to contact fragile parts of the femur itself and so damage to the femur is minimised.

Similarly, the distracting force may be applied to the tibia through a trial tibial component thereby preventing damage to the fragile surface of the tibia itself. In addition, the distraction procedure does not rely on the position of the cutting guide to obtain the desired tension. This results in the positioning of the cutting guide being independent of the distraction procedure, which leads to a greater degree of freedom when positioning the cutting guide.

Optionally, the method of the fifth aspect of the invention further comprises the step of coupling the distraction device to the intramedullary rod prior to the step of operating the distraction device. The method may further comprise the step of coupling the distraction device to a trial tibial component prior to the step of operating the distraction device.

In a particular embodiment, the method of the fifth aspect of the invention further comprises the step of positioning a cutting guide adjacent the end of the femur. Optionally, the method of the fifth aspect of the invention further comprises the step of adjusting the relative positioning of the cutting guide with respect to the femur. The method of the fifth aspect of the invention may further comprise the step of inserting a spacer between the tibia and the cutting guide, the spacer being sized to obtain the desired flexion gap. In addition, the method of the fifth aspect of the invention may further comprise the step of determining whether the cutting guide extends to the desired position with respect to the anterior of the femur when the cutting guide is allowed to rest on the spacer and, if not, replacing the cutting guide with a different sized cutting guide to achieve the desired position. Optionally, the method of the fifth aspect of the invention further comprises the step of measuring the degree of rotation between the cutting guide and the femur. The method may then comprise the steps of soft tissue release and repetition of the above method steps if the degree of rotation between the cutting guide and the femur is not in the desired range. The method may then comprise the step of repeating all of the above steps until the degree of rotation between the cutting guide and the femur is in the desired range.

### BRIEF DESCRIPTION OF THE DRAWINGS

The invention will now be described, by way of example, with reference to the accompanying drawings in which:

FIG. 1a illustrates an intramedullary rod for use in embodiments of the present invention;

FIG. 1b illustrates the intramedullary rod of FIG. 1a with a handle in position;

FIGS. 2a and 2b show two components of a distraction device for use in embodiments of the present invention;

FIG. 3a shows the components of FIGS. 1a, 2a and 2b assembled together;

FIG. 3b shows the positioning of a screwdriver for extending/contracting the length of the distraction device of FIGS. 2a and 2b;

FIG. 4a is a front elevational view of a cutting guide for use in embodiments of the present invention;

FIG. 4b is a rear elevational view of the cutting guide of FIG. 4a;

FIG. 4c is a view similar to that of FIG. 4b showing the cutting guide being located over the assembly of FIG. 3a;

FIG. 4d is a view similar to that of FIG. 4c showing the cutting guide being locked onto the assembly of FIG. 3a;

FIG. 5a is a perspective view of the instrumentation of FIGS. 1a to 4d in use;

FIG. 5b shows adjustment of the distraction device during use to establish the desired tension in the collateral ligaments;

FIG. 6 shows insertion of spacers and the temporary pinning of the cutting guide to a femur during use;

FIG. 7 is a perspective view of an ‘anti-notch’ guide which may be used in embodiments of the present invention;

FIG. 8 illustrates use of the ‘anti-notch’ guide of FIG. 7;

FIG. 9 is a perspective view of an ‘external rotation protractor’;

FIG. 10 illustrates the use of the ‘external rotation protractor’ of FIG. 9 in an embodiment of the present invention;

FIG. 11a is a front perspective view of an alternative ‘external rotation protractor’ to that of FIGS. 9 and 10, showing the positional relationship between the protractor and the cutting guide;

FIG. 11b is a rear perspective view of the protractor and cutting guide of FIG. 11a;

FIG. 12 illustrates the fixing of the cutting guide to the femur after determination of size and location in accordance with embodiments of the present invention;

FIG. 13 illustrates a tibial component according to a further aspect of the present invention;

FIG. 14 is a sectional view of the tibial component of FIG. 13; and

FIG. 15 illustrates a device for coupling the distraction device of FIGS. 2a and 2b to a trial tibial component.

### DETAILED DESCRIPTION OF CERTAIN EMBODIMENTS

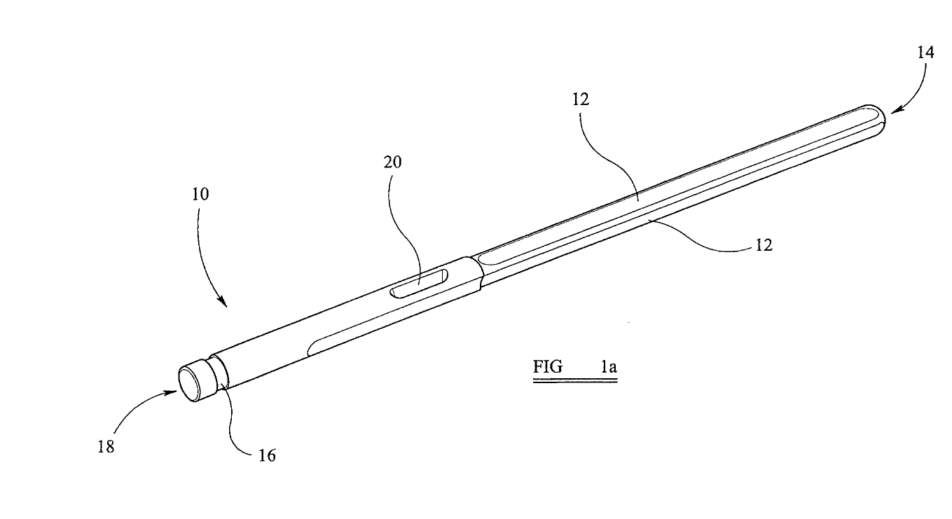

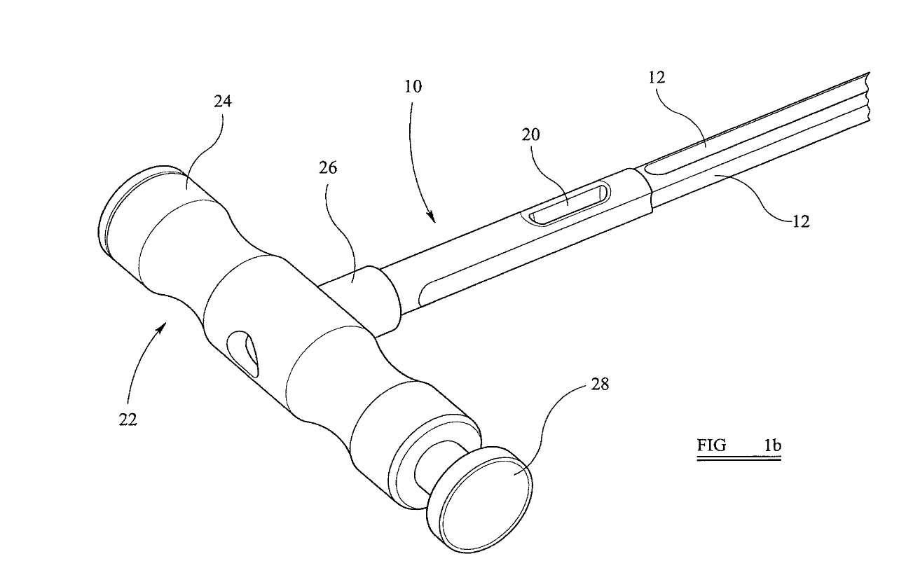

With reference to FIG. 1a, there is illustrated an intramedullary rod 10 according to the present invention. The rod 10 is made from metal and is substantially cylindrical. Four equi-spaced longitudinal grooves 12 extend along the rod 10 from a first end 14. An annular groove 16 is provided a short distance from a second end 18 of the rod 10. An aperture 20 is provided approximately a third of the way along the length of the rod 10 from the second end 18. The aperture 20 passes orthogonally through the longitudinal axis of the rod 10. The aperture 20 has a width substantially equal to the width of the longitudinal grooves 12 and is disposed such that it is aligned with a respective longitudinal groove 12 on either side of the rod 10. The aperture 20 has a length substantially greater than its width so as to form an elongate slot. The two longitudinal grooves 12 that are aligned with the aperture 20 terminate close to it while the other two longitudinal grooves 12 extend to a point approximately midway between the aperture 20 and the second end 18.

As shown in FIG. 1b, the intramedullary rod 10 is provided with a handle 22. The handle 22 is configured for attachment to the second end 18 of the rod 10. The handle 22 is generally T-shaped with a substantially cylindrical hollow crossbar 24 and a hollow cylindrical sleeve 26 constituting the upright of the ‘T’.

The second end 18 of the rod 10 is inserted in the sleeve 26 such that the annular groove 16 is disposed in the centre of the crossbar 24 and the longitudinal grooves 12 extend away from the sleeve 26. A bolt 28 is provided through one end of the crossbar and tightened to contact the annular groove 16 and lock the handle 22 and rod 10 together. The crossbar 24 of the handle 22 is positioned with its longitudinal axis at 90° to the axis of the aperture 20 in the rod 10 before the handle 22 is locked to the rod 10. This allows the surgeon to determine the relative orientation of the aperture 20 through the rod 10 by simply observing the orientation of the handle 22 with respect to the rod 10.

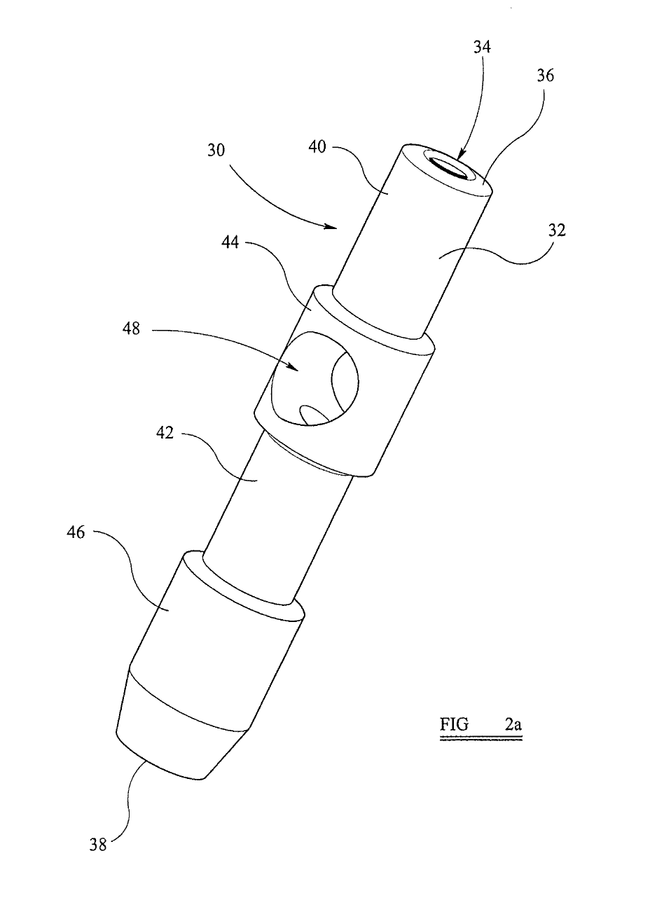

Relatively displaceable parts (members) of a distraction device according to the present invention are shown in FIGS. 2a and 2b. A first part (member) 30, shown in FIG. 2a, has a tubular body 32 with a longitudinal bore 34 passing from an upper end 36 to a lower end 38. The body 32 is composed of four cylindrical sections with the first (upper) and third sections 40, 42 having the same diameter and the second and fourth (lower) sections 44, 46 having enlarged diameters. The second section 44 includes a cylindrical aperture 48 with an axis that intersects that of the longitudinal bore 34 and is orientated perpendicularly thereto. The fourth section 46 is inwardly tapered at the lower end 38 of the body 32 and the internal bore 34 in the fourth section 46 is threaded (not shown).

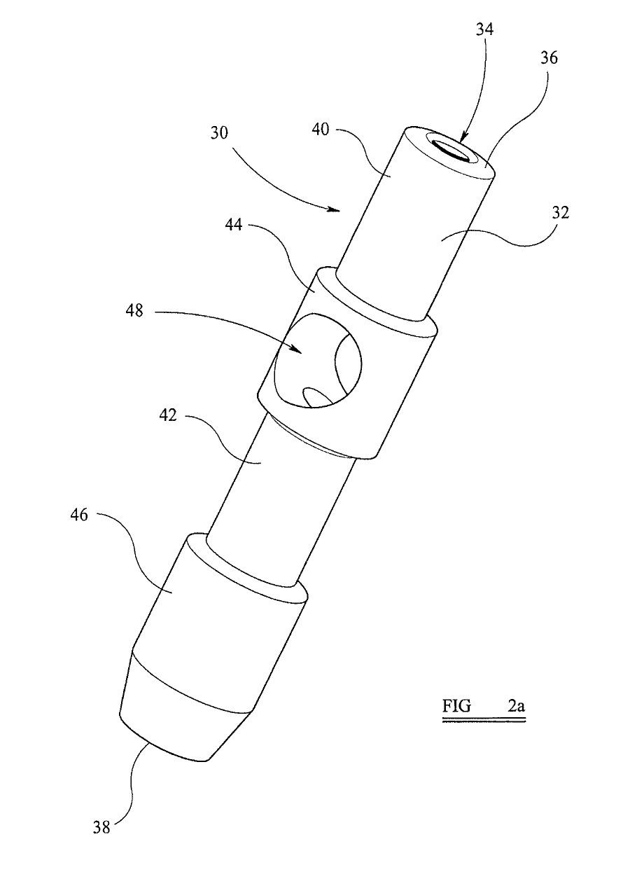

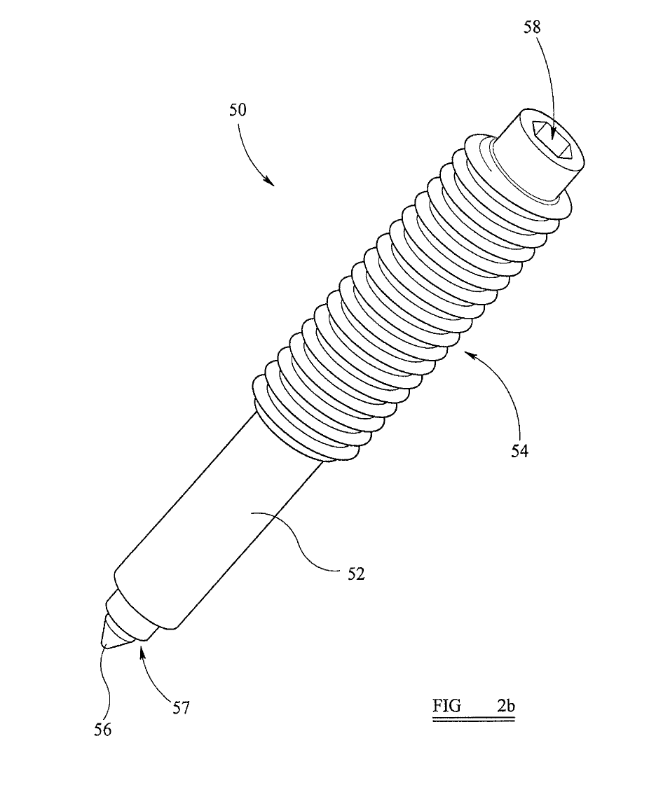

A second part (member) 50 of the distraction device, shown in FIG. 2b, is configured for relative movement with respect to the first part 30. The second part 50 is in the form of a screw and comprises a cylindrical shank 52 externally threaded over its upper half 54 and tapered at a lower end to form a tip 56. Towards the tip 56 there is provided a shoulder 57 in the form of an inwardly directed perpendicular step in the shank 52. The shoulder 57 is configured to stabilise the distraction device when used on a trial tibial component as described later. A hexagonal recess 58 is provided in an upper end of the shank 52 for receiving the hexagonal tip of a screwdriver 60 (shown in FIG. 3b).

FIG. 3a shows the first and second parts 30, 50 of FIGS. 2a and 2b engaged to form the distraction device 62 of the present invention. Thus, the screw thread of the second part 50 is screwed into the bore 34 in the forth section 46 of the first part 30 such that the tip 56 and lower portion of the shank 52 extends from the body. The distraction device 62 is secured to the intramedullary rod 10 of FIG. 1a by passing the rod 10 through the aperture 48 in the second section 44 of the body 32 until the aperture 20 in the rod 10 intersects the bore 34 of the distraction device 62.

The relative displacement of the first and second parts 30, 50 of the distraction device 62 can be adjusted by insertion of a tool, in the form of a hexagonal screwdriver 60, through the bore 34 to engage in the complementary shaped recess 58 in the second part 50, as shown in FIG. 3b. The aperture 20 in the rod 10 allows the screwdriver 60 to pass through the bore 34 when the distraction device 62 is engaged on the rod 10. Rotating the screwdriver 60 about its longitudinal axis results in the second part 50 being screwed into or out of the lower end of the first part 30 of the distraction device 62 and so the distance between the intramedullary rod 10 and the tip 56 can be varied.

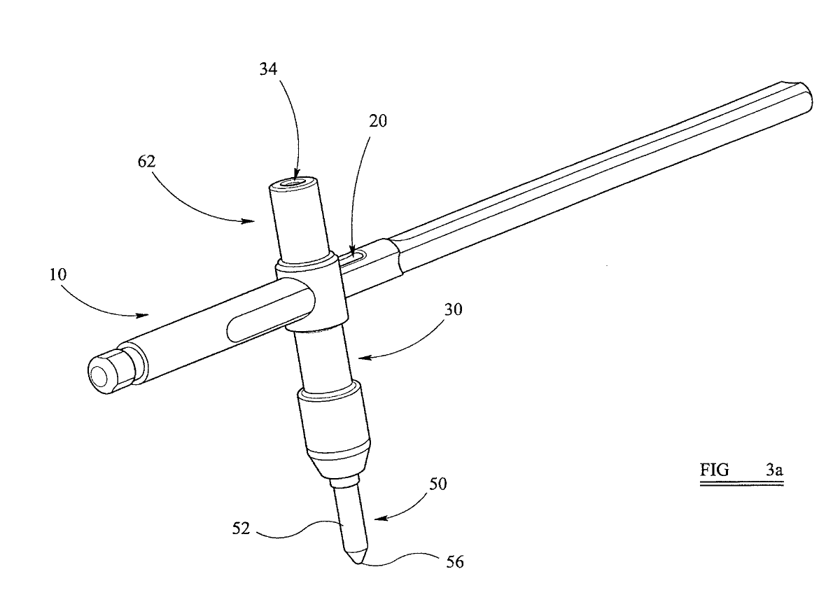

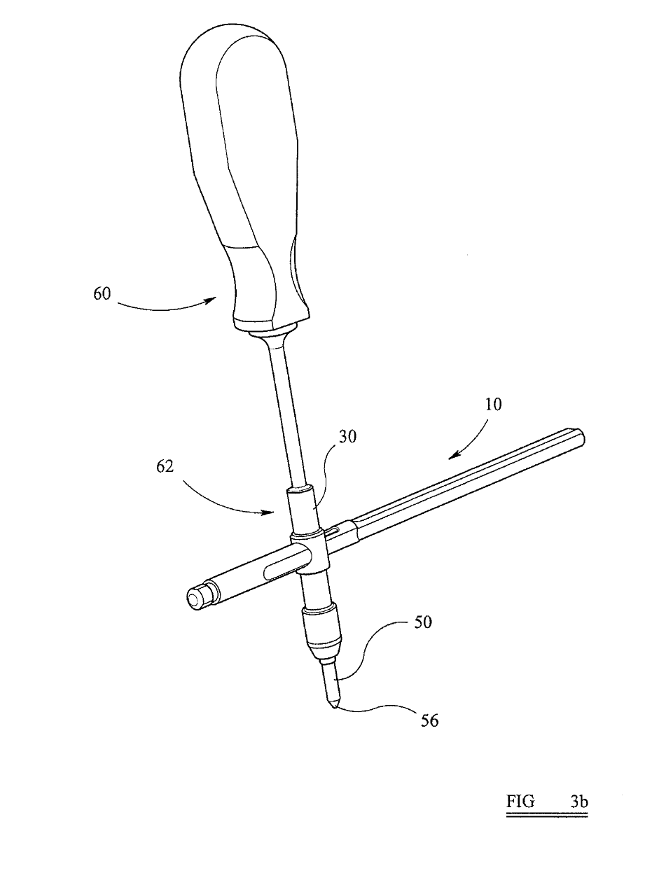

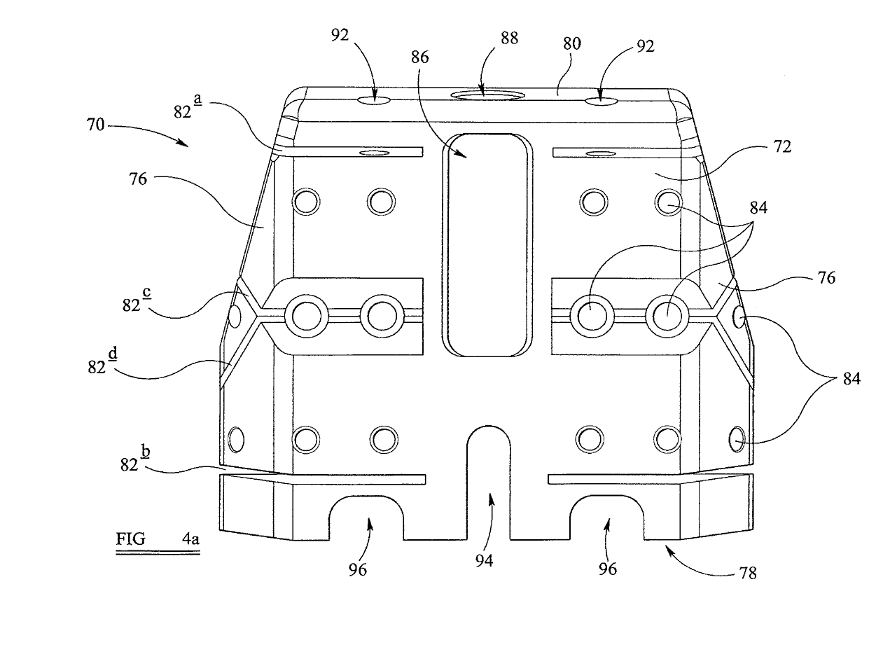

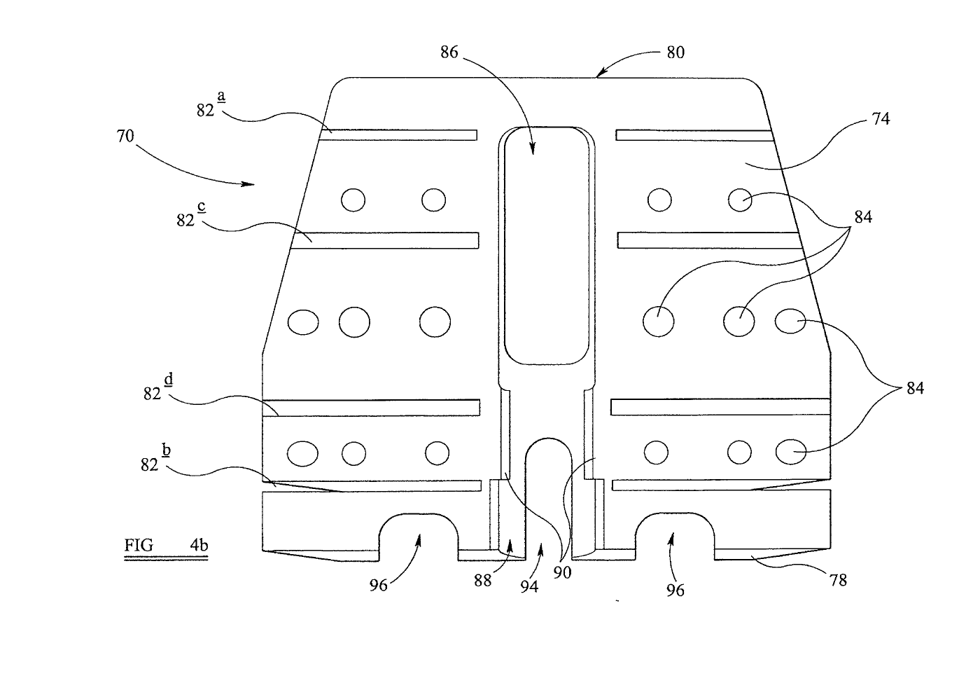

FIGS. 4a through 4d show a cutting guide 70 according an aspect of the present invention. The cutting guide 70 is generally in the form of a rectangular block with a front face 72, a rear face 74, two sidewalls 76, a base 78 and a top 80. The Applicant intends to market this component under the trademark the ‘Birmingham Block’.

The cutting guide 70 includes a plurality of slits 82 passing from the front face 72 of the guide 70 to the rear face 74 of the guide 70. The slits 82 are configured to permit the passage of blades (not shown) therethrough to form the cuts required to produce a resected distal end of a femur with appropriately angled planar surfaces to mate with the interior surface of a prosthetic femoral component (not shown). In the embodiment illustrated, an upper horizontal slit 82a, a lower horizontal slit 82b, a central upwardly inclined slit 82c and central downwardly inclined slit 82d are provided on each side of the guide 70. The upwardly and downwardly inclined slits 82c, 82d are arranged such that they bisect each other at approximately the centre of the guide 70. Note, in this particular embodiment, the distance between the base 78 and the lower horizontal slits 82b is configured to equal the thickness of the posterior condyles of the prosthetic femoral component to be employed (not shown).

The cutting guide 70 also includes several holes 84 configured for fixing pins or screws to pass therethrough to secure the cutting guide 70 to the distal end of a femur. In the present embodiment, a large number of holes 84 are provided to allow the surgeon the choice of the most appropriate holes 84 to use in any particular situation. Thus, there are provided a pair of horizontally aligned upper, central and lower holes 84 on each side of the guide. There are also two vertically spaced holes 84 angled from either sidewall 76 to the rear face 74 of the guide 70. The four central holes 84 pass through the intersection of the upwardly and downwardly inclined slits 82c 82d.

An upper central vertically elongate slot 86 is provided through the cutting guide 70 to allow the intramedullary rod 10 to pass through from the rear face 74 to the front face 72. The elongate nature of the slot 86 permits the cutting guide 70 to be moved upwardly and downwardly (or anteriorly and posteriorly) when located over the intramedullary rod 10. The slot 86 is closed at its upper and lower ends to limit the travel of the cutting guide 70 with respect to the intramedullary rod 10. However, the length of the slot 86 is such that the extreme positions of the rod 10 and cutting guide 70 fall outside the functional range and so the anterior and posterior movement of the cutting guide 70 is essentially unconstrained.

A substantially cylindrical channel 88 extends centrally through the cutting guide 70 from its top 80 to its base 78. As can be seen from FIGS. 4b, 4c and 4d the channel 88 intersects the elongate slot 86 and is open on the rear face 74 from the base 78 to a position close to the top 80. At approximately a quarter of the way up the rear face 74 from the base 78, the channel 88 includes two opposed lips 90 curving outwardly from the rear face 74 and extending a short distance around the channel 88. As shown in FIG. 4c, the lips 90 allow passage of the narrower sections of the distraction device 62 (i.e. the first and third sections 40, 42) to pass therethrough—permitting insertion of the distraction device 62 into the channel 88. However, once the distraction device 62 has been inserted in the channel 88 and the cutting guide 70 is slid posteriorly so that the first section 40 of the distraction device 62 extends through the top 80 of the cutting guide 70, the fourth section 46 of the distraction device 62 is disposed adjacent the lips 90 (see FIG. 4d). As the fourth section 46 is wider than the third section 42 the fourth section 46 is unable to pass through the lips 90 and the distraction device 62 is therefore retained within the channel 88.

As shown in FIG. 4a, the top 80 of the cutting block 70 includes two vertically extending apertures 92, one on either side of the channel 88. These are provided for engagement with an anti-notch guide as will be described later.

As can be seen from FIGS. 4a and 4b, a central notch 94 is provided from the base 78 of the cutting guide 70 extending approximately a quarter of the way up the guide 70. A cut-out 96 from the base 78 is provided to either side of the central notch 94. The notch 94 and cut-outs 96 are provided for engagement with an external rotation protractor as will be described later.

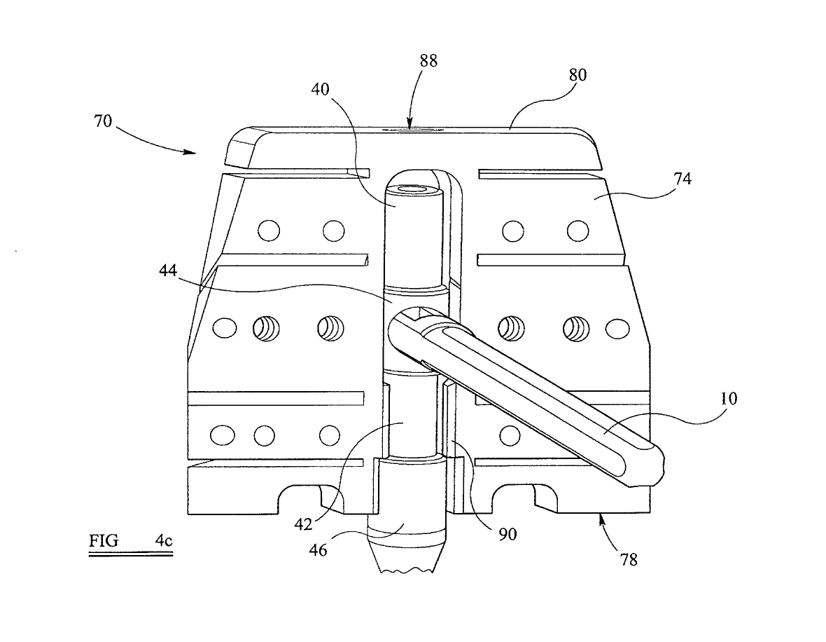

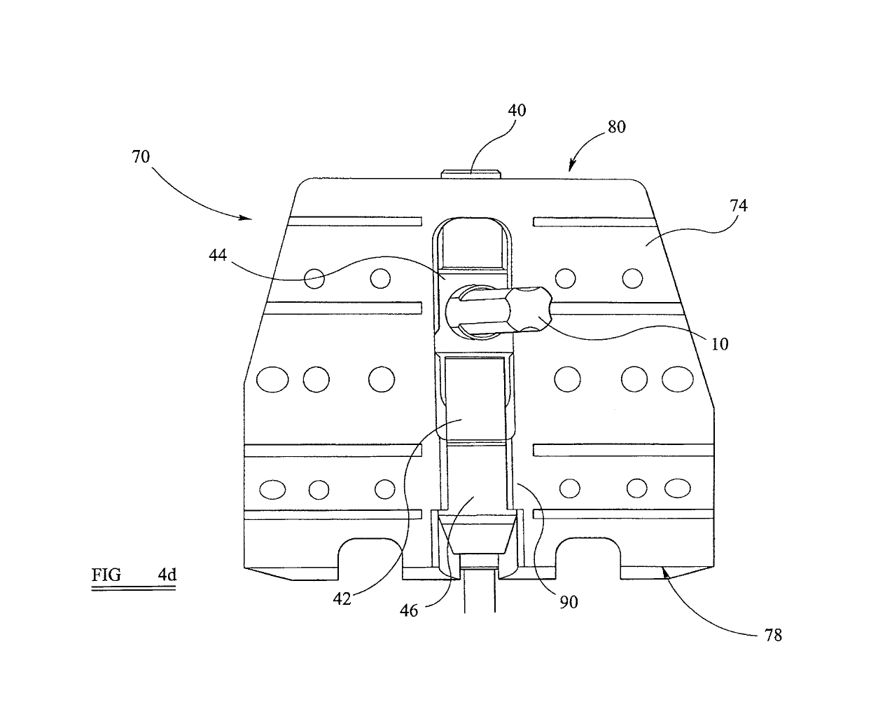

FIG. 5a illustrates use of the instruments of FIGS. 1a through 4d during TKR surgery. The distal end of a patient's femur 100 is shown adjacent a trial tibial component 102 disposed as though the knee is bent by 90°. Prior to this illustration the proximal tibial bone resection would have been performed and the distal end of the femur 100 would have been resected with a vertical distal cut. The first end of the intramedullary rod 10 of FIG. 1a would then have been inserted into the medullary canal of the femur 100 so as to leave the aperture 20 in the rod 10 just exposed. The distraction device 62 of FIG. 3a would then have been engaged on the rod 10 by sliding the second end 18 of the rod 10 through the cylindrical aperture 48 in the second section 44 of the body 32 of the distraction device 62 until the aperture 20 in the rod 10 intersects the longitudinal bore 34 in the distraction device 62. The cutting guide 70 of FIGS. 4a and 4b would then have been located over the intramedullary rod 10 and distraction device 62 through the sequence illustrated in FIGS. 4c and 4d. In the resulting configuration shown, the intramedullary rod 10 and first part 30 of the distraction device 62 are held in a fixed relationship with the femur 100 while the cutting guide 70 can be moved vertically (i.e. in an anterior-posterior direction) with respect to the femur 100. In addition, the second part 50 of the distraction device 62 can be moved relative to the first part 30 through use of a screwdriver 60, as shown in FIG. 5b and described previously in relation to FIG. 3b.

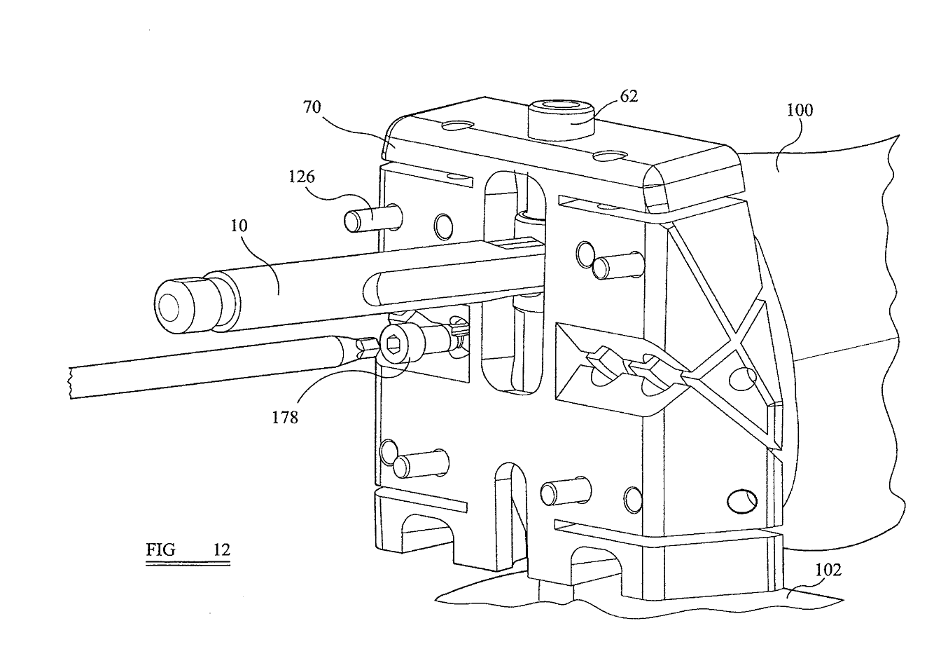

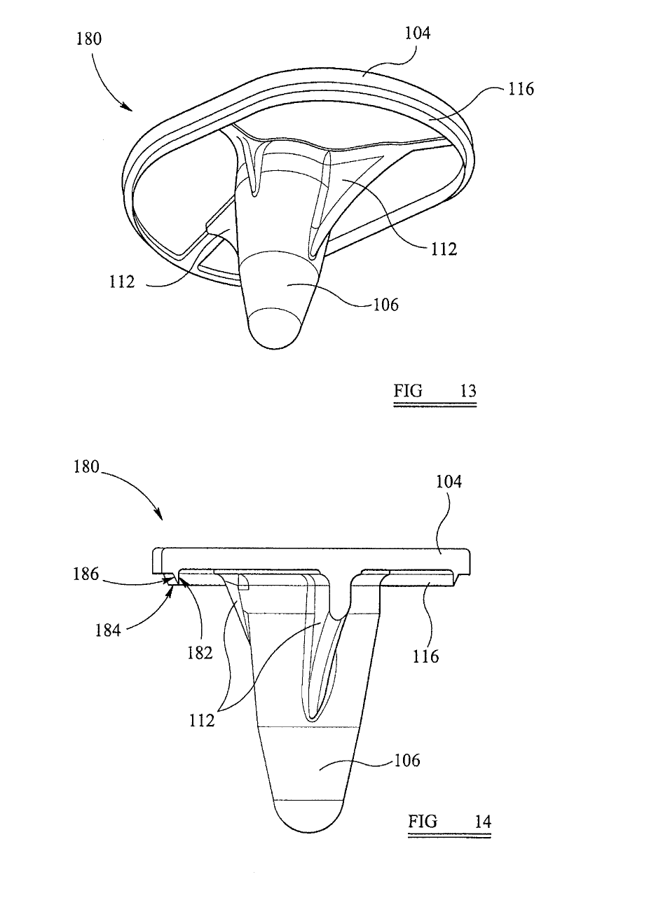

The trial tibial component 102 shown in the present embodiment comprises a plateau 104 with a distal surface configured for mating with a proximal end of a resected tibia (not shown) and a conical locating stem 106 extending from the distal surface for insertion into the medullary canal of the tibia. The plateau includes a central circular aperture 108 configured to receive a stem of a bearing component (not shown) and a distraction slot 110 extending radially outwardly in an anterior direction towards the edge of the plateau 104 from close to the circular aperture 108. The distraction slot 110 allows the femur 100 to reach a comfortable anterior-posterior position with respect to the tibia when the collateral ligaments are tensioned. The locating stem 106 is provided with three radially spaced triangular wings 112 extending vertically downwardly from the distal surface of the plateau 104. The two wings 112 on the medial and lateral sides of the tibia are significantly larger than the wing 112 on the anterior side of the tibia. Each wing 112 includes serrations 114 along its free edge for cutting wing shaped slots in the proximal end of the tibia (not shown). These slots are then able to receive non-serrated smooth edged wings of a prosthetic tibial component, such as that shown in FIGS. 13 and 14. An annular protrusion 116 (better shown in FIGS. 13 and 14 and described in more detail later) extends from the distal surface of the plateau 104 to aid in attachment of the tibial component 102 to the tibia.

As described earlier in relation to FIG. 1b, the handle 22 may be attached to the intramedullary rod 10 to facilitate rotation of the rod 10 and alignment of the aperture 20 in the rod 10 with the bore 34 of the distraction device 62. This allows for insertion of the screwdriver 60 through the bore 32 for operation of the distraction device 62, as shown in FIG. 5b.

As can be seen in FIGS. 5a and 5b, during use, the tip 56 of the distraction device 62 is located in the distraction slot 110 of the trial tibial component 102 such that the shoulder 57 of the second part 50 rests upon the surrounding plateau 104. This helps to keep the second part 50 perpendicular to the plateau 104. When a distracting force is applied between the intramedullary rod 10 and the trail tibial component 102 it is desirable that the second part 50 remains perpendicular to the plateau 104 when viewed from the anterior of the tibia. Such an arrangement allows for equal tensioning of the collateral ligaments and soft tissue structures on either side of the knee due to the fact that the femur 100 is free to rotate relative to the tibia. To allow such femoral rotation either the intramedullary rod 10 rotates relative to the distraction device 62 or the femur 100 rotates relative to the intramedullary rod 10. It is undesirable for relative rotation to occur between the femur 100 and tibia due to angulation between the second part 50 and the trial tibial component 102 when viewed from the anterior of the tibia. It is possible for both of the above situations to occur simultaneously. If any angulation does occur between the second part 50 and the trial tibial component 102 then the cutting guide 70 can be manually rotated on the femur 100 to keep the second part 50 perpendicular. Operation of the distraction device 62 in this configuration alters the spacing between the trail tibial component 102 and the femur 100 and allows for an equal collateral ligament tension to be obtained.

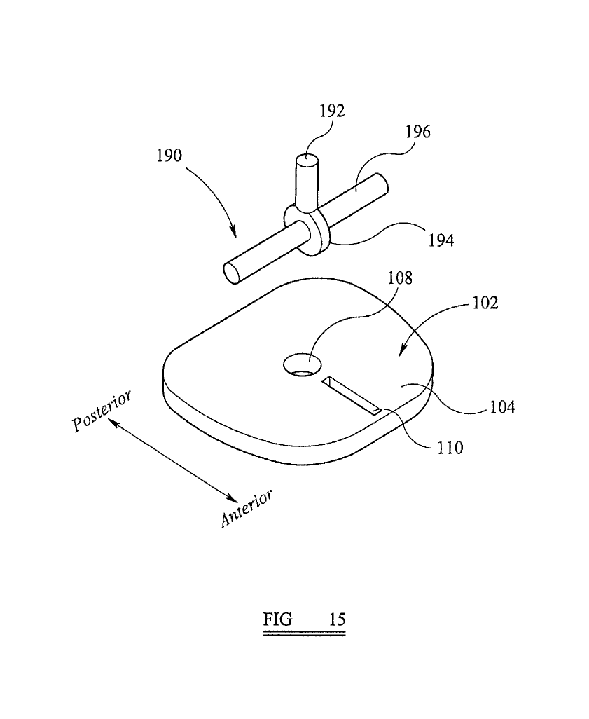

Alternative constructions for keeping the second part 50 perpendicular to the trial tibial component 102 are as follows. The free end of the second part could be blunt and the trial tibial component could be provided with a hole (say, 3 mm deep in a plateau 4 mm thick) sized to receive the blunt end. In this embodiment, however, the second part will not be allowed to find its correct anterior-posterior position on the trial tibial component, it could be easily damaged or broken if the knee is hyper-flexed, and with continual use, it is likely that the edges of the hole will wear and deform permitting angulation. Instead of a hole, a channel with perpendicular sides could be provided in the position of the slot 110. This would over come the first two disadvantages of the arrangement with the hole but the problem of wear would still exist. Accordingly, the Applicant has devised a device 190, shown in FIG. 15, for coupling the distraction device 62 to the trial tibial component 102. Note that in this Figure only the plateau 104 of the trial tibial component 102 is shown for clarity but in practice the trial tibial component 102 would include all features of that described above in relation to FIG. 5a. The device 190 includes a hollow cylindrical socket 192 for receiving the free end of the second part 50. The socket 192 is radially mounted on a wheel 194. The wheel 194 is configured for rotation in a channel (i.e. slot) 110 of the trial tibial component 102 and is rotatably mounted on an axle 196. The axle 196 is configured for rolling along the plateau 104 of the trial tibial component 102. Thus, the device 190 permits adjustment of the distraction device 62 in the anterior and posterior directions of the tibia. The device 190 is also capable of holding the second part 50 perpendicularly to the proximal end of the tibia (as viewed from the anterior) due the tight engagement of the wheel 194 in the channel 110 and the fact that the axle 196 rests on the plateau 104 during use. Furthermore, because of the wheel 194 the device 190 permits flexion and/or extension of the knee during use.

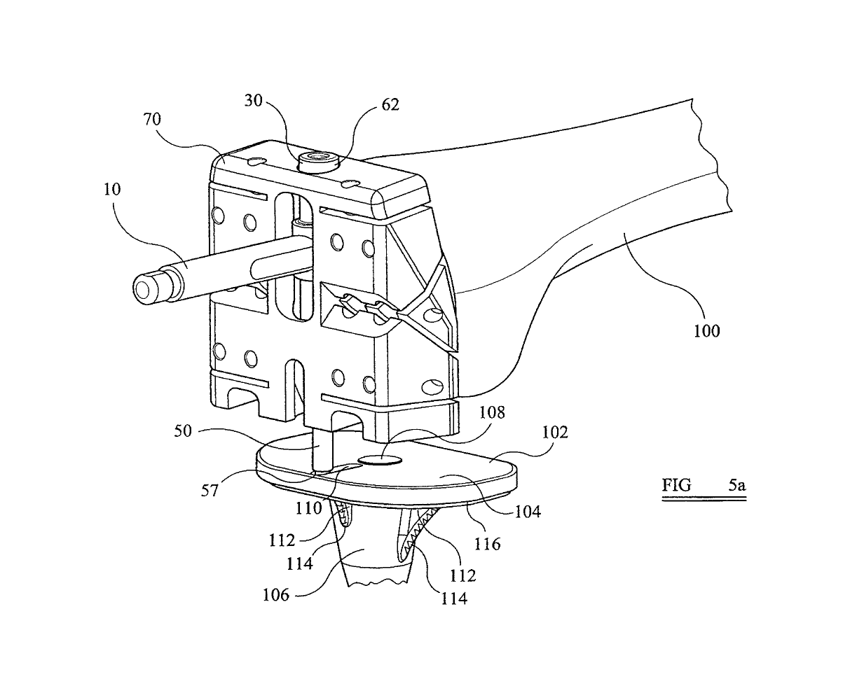

Once the desired collateral ligament tension has been obtained, two spacers 120 are positioned on the plateau 104 of the tibial component 102 and the base 78 of the cutting guide 70 is allowed to rest thereon by sliding the cutting guide 70 down the distraction device 62, as shown in FIG. 6. The spacers 120 are essentially cylindrical blocks with a loop of material 122 emanating from their respective sides 124 to facilitate in their removal. The spacers 120 are provided in a range of nominal sizes (e.g. 10, 12.5, 15, 17.5 and 20 mm in height) so that the appropriate size can be selected to provide the desired flexion gap for each patient. Note that since, in the embodiment illustrated, the base 78 of the cutting guide 70 is configured to represent the base of the posterior condyles of the prosthetic femoral component to be employed, the size of spacers 120 can be chosen, taking into account the relative thicknesses of the trial tibial component and the prosthetic tibial component and bearing component, to provide the desired flexion gap.

As also shown in FIG. 6, temporary pins 126 can be inserted into the desired fixing holes 84 in the cutting guide 70 once the spacers 120 are in place and the cutting guide 70 is resting thereon. However, in accordance with a further aspect of the present invention, it is best to determine whether the correct size of cutting guide 70 is being used before any temporary pins 126 are inserted.

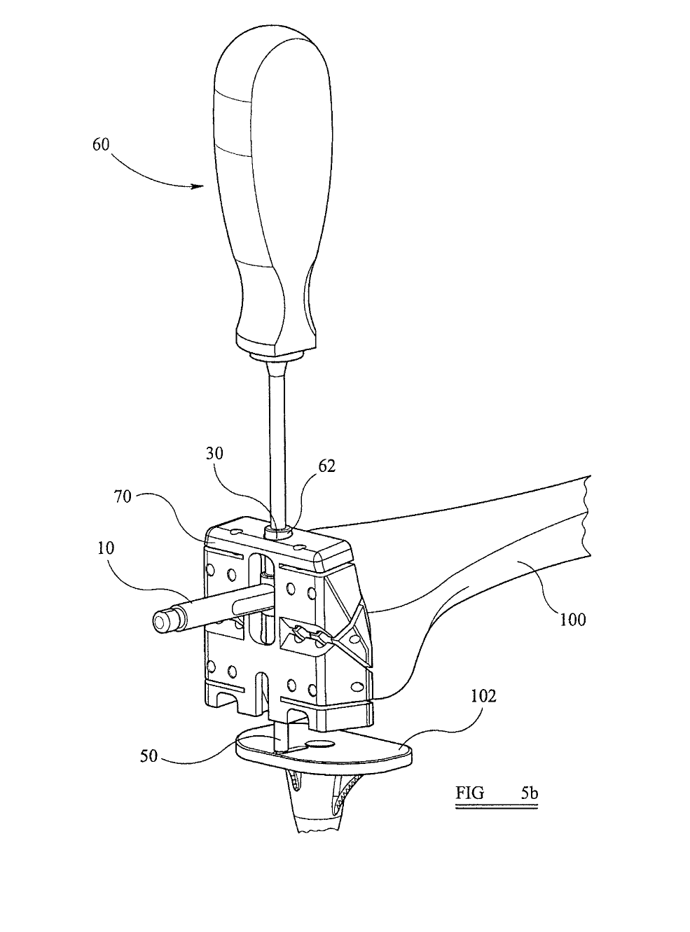

Accordingly, it is preferable to use an anti-notch guide 130 as shown in FIG. 7. The anti-notch guide 130 includes two vertically disposed locating elements 132 for respective engagement in the two vertically extending apertures 92 on the top 80 of the cutting guide 70. The two locating elements 132 are mounted perpendicularly on respective ends of a U-shaped yoke 134. Extending from the centre of the yoke 134, in the opposition direction to the ends of the yoke 134, is an elongate shaft 136. An indicator 138, or spacer element, is mounted perpendicularly at the free end of the shaft 136. In the embodiment shown, the indicator 138 comprises a rectangular metal plate with a curved cut-out 140 in each side wall 142. The indicator 138 is mounted with the shaft 136 positioned off-centre and towards one of the cut-outs 140. An aperture 144 is provided in the indicator 138 between the shaft 136 and the other of the cut-outs 140.

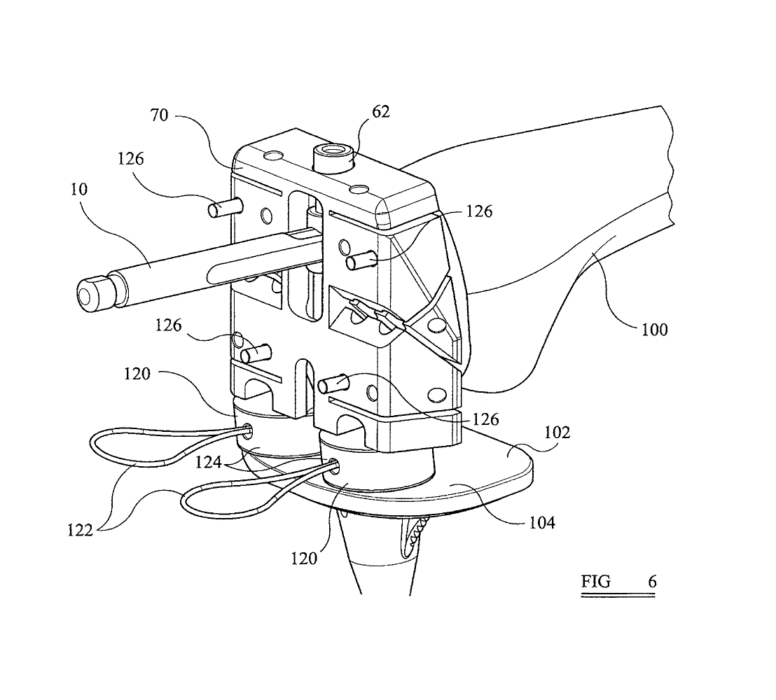

As shown in FIG. 8, when the anti-notch guide 130 is mounted on the cutting guide 70, the yoke 134 rests on the top 80 of the cutting guide 70 and the shaft 136 extends generally in the direction of the femur 100 so that the indicator 138 is disposed to the anterior of the femur 100. The position of the indicator 138 relative to femur 100 can provide an indication as to whether the correct size of cutting guide 70 is being used. For example, if there is a large gap between the indicator 138 and the femur 100 when the cutting guide 70 is resting on the spacers 120, then the cutting guide 70 is too big and should be replaced by a smaller cutting guide 70 such that the gap is minimised. If there is not enough space for the anti-notch guide 130 to be correctly positioned on the cutting guide 70 (i.e. because the indicator 138 contacts the femur 100 before the yoke 134 is allowed to rest on top 80 of the cutting guide 70), then the cutting guide 70 is too small and should be replaced by a larger cutting guide 70 such that the anti-notch guide 130 can be correctly positioned.

Once the correct size of cutting guide 70 has been selected and placed on the spacers 120, temporary pins 126 can be inserted into the desired fixing holes 84 as described in relation to FIG. 6.

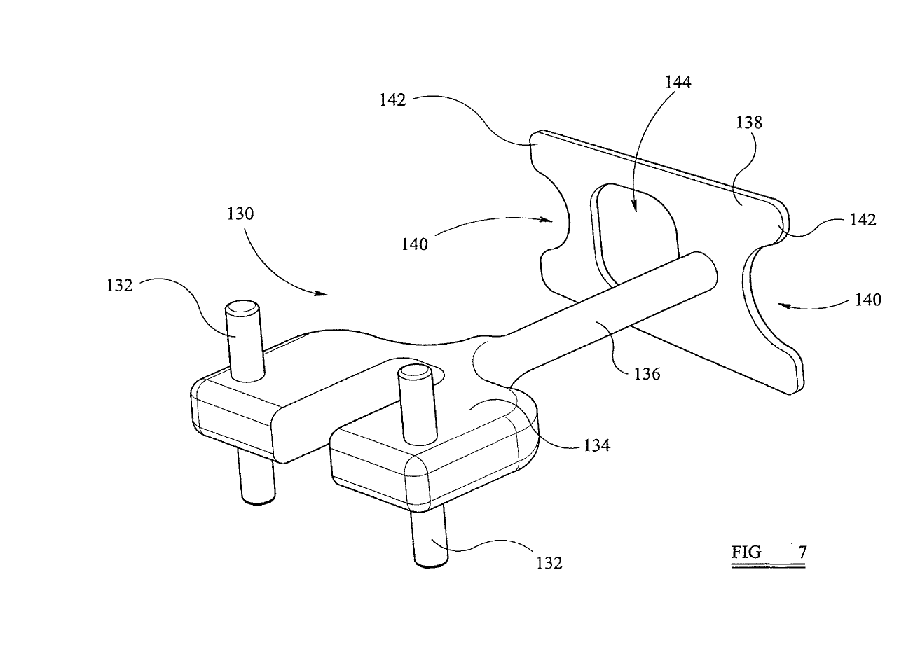

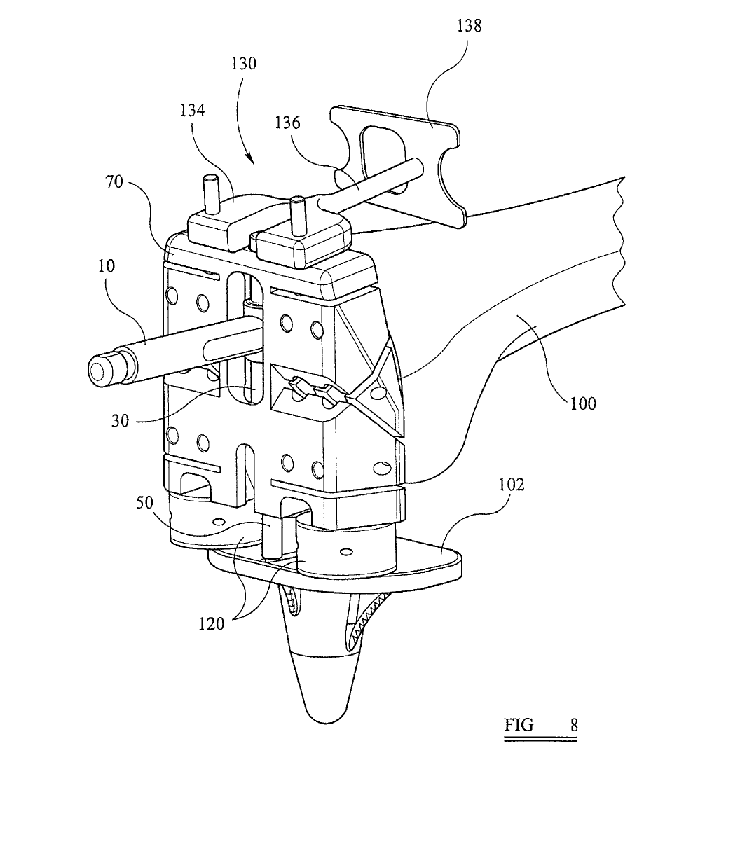

The next part of the procedure ensures that the cutting guide 70 is in the desired rotational alignment with the femur 100. To minimise the risk of patella mal-tracking on a prosthetic femoral component, it is preferable for the cutting guide 70 to be positioned at an angle of between 2° to 7° of external rotation. Internal rotation is particularly undesirable. Traditionally, a surgeon simply adjusts the angle of the cutting guide 70 by eye. However, in the present invention, an external rotation protractor 150 such as that shown in FIG. 9 is employed. This rotation protractor 150 includes a pole 152 with a reference plate 154 perpendicularly mounted at one end. The reference plate 154 has a front face 156, connected to the pole 152, which is marked with a scale 158. A rear face 160 of the reference plate 154 is provided with a locating element (not shown) configured to locate within the central notch 94 of the cutting guide 70, as shown in FIG. 10.

Two horizontally spaced paddles 162 are harnessed together by a square frame 164 which is rotationally mounted on the pole via a collar 166 positioned adjacent the front face 156. The paddles 162 are configured to extend beneath the lateral and medial condyles of the femur 100 when the rotation protractor 150 is engaged with the cutting guide 70, as described above. The collar 166 is provided with a pointer 168 configured for referencing the scale 158. Relative rotation of the paddles 162 with respect to the reference plate 154 is therefore indicated on the scale 158.

As shown in FIG. 10, in use, the rotation protractor 150 is positioned such that the paddles 162 contact the posterior of the lateral and medial condyles, respectively, while the reference plate 154 is held in fixed relationship with the cutting guide 70. Accordingly, the relative angular displacement between the condyles and the cutting guide 70 can be measured.

If the angle of the cutting guide 70 relative to the femur is not in the desired range it is not simply a matter of rotating the cutting guide 70 to taste. If that were done without any other measures taken it would result in unequal tension of the collateral ligaments and soft tissue structures on the medial and lateral sides of the knee. This is undesirable for the reasons mentioned previously (i.e. patient instability and risk of dislocation). Consequently, an additional procedure involving soft tissue release is required. Such procedures are known although the soft tissue release procedure required to alter the rotational position of the cutting guide 70 in flexion is not as common as the soft tissue release procedure performed in extension to obtain a rectangular extension gap.

The overall aim of the various aspects of the present invention is to allow for the cuts on the bones and hence the position of the implants to be in harmony with the collateral ligaments and other soft tissue structures that hold the bones together.

In principle a soft tissue release is required if the soft tissue envelope on one side of the knee is shorter than on the other side. This occurs commonly in osteoarthritis of the knee and the common type is medial arthritis. In such a case, the cartilage and bone on the medial side of the knee is worn away and the medial soft tissue envelope slowly contracts over the years. This leads to a varus, or bow, leg deformity. When TKR surgery is performed on such an arthritic knee it is possible to carry out the procedure and leave the varus deformity uncorrected. This is the easy option for the surgeon since no soft tissue release is performed, however, it is not good for the patient because the leg will still be deformed and unacceptable stresses will be placed on the medial side of the knee. Accelerated wear of the medial side of the bearing component, fracture of the tibial component and loosening of the implants in their bony beds have all been associated with uncorrected varus deformity.

In principle soft tissue release involves the lengthening of the soft tissues on the shortened side of the knee to match those on the longer side. It is most commonly performed by an incremental partial disconnection of the soft tissue envelope from either the tibia or the femur on the tight side. However, it will be understood that the method of soft tissue release is not important for the purposes of the present invention.

After a soft tissue release has been performed the various steps described above are repeated (i.e. the tensioning, the locating of the cutting guide 70, the insertion of spacers 120 to set the desired flexion gap, the checking that the cutting guide 70 is the correct size, the temporary pinning of the cutting guide in place and the measuring of the rotational alignment). By this stage it is hoped that the angle of rotation is in the desired range, however, if not, a further soft tissue release is performed and the above steps repeated until the angle is in the desired range.

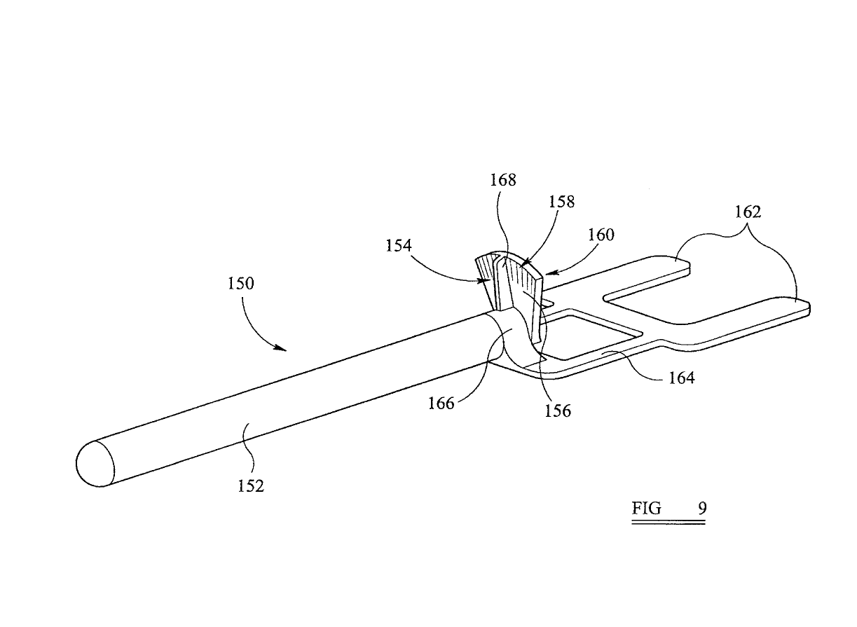

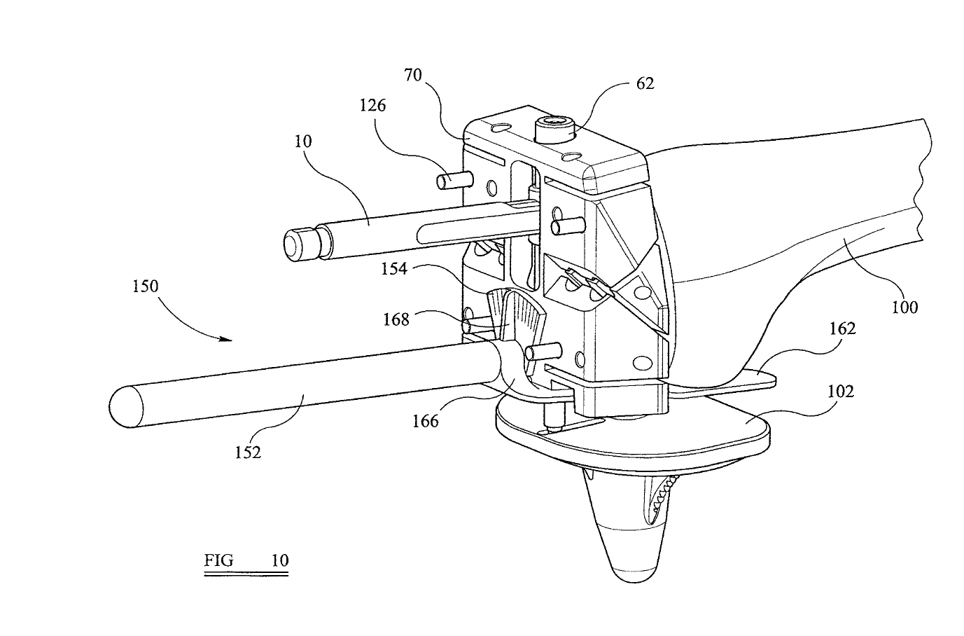

An alternative external rotation protractor 170 is shown in FIGS. 11a and 11b with like parts numbered accordingly. This embodiment differs from that of FIGS. 9 and 10 in that the paddles 162 are harnessed together via a U-shaped yoke 172 as opposed to a square frame 164. This allows for easy insertion of the rotation protractor 170 beneath the cutting guide 70, even when the distraction device 62 is still in place. In addition, the ends of the yoke 172 include upturned flanges 174 from which the paddles 162 extend so that the paddles 162 are disposed in a plane parallel to but vertically spaced from that of the yoke 172.

FIG. 11b shows a locating element 174 on the rear face 160 of the reference plate 154 engaging within the central notch 94 of the cutting guide 70. A similar locating element 174 is provided in the embodiment of FIGS. 9 and 10 although not shown. FIG. 11b also shows alignment of the arms of the yoke 172 with the cut-outs 96 of the cutting guide 70. Accordingly, the cut-outs 96 allow for rotation of the yoke 172 relative to the cutting guide 70.

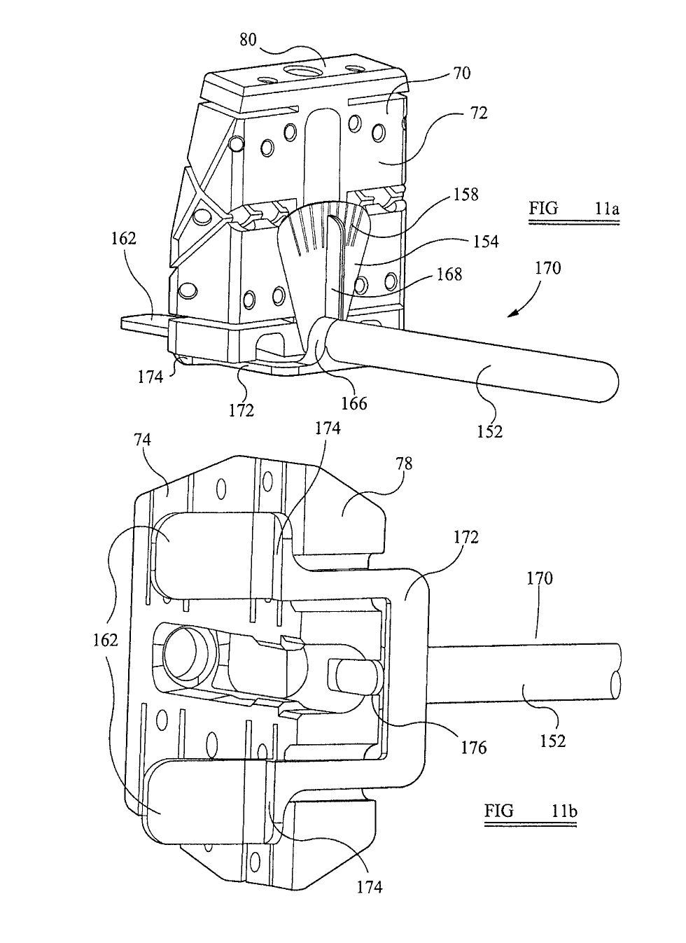

Once the cutting guide 70 is in the correct rotational alignment it is secured to the femur 100 by placing screws 178 through the desired holes 84, as indicated in FIG. 12. Once secure, the temporary pins 126 may be removed before cutting blades (not shown) are inserted into the various slits 82 in the cutting guide 70 to perform the required cuts. The remainder of a TKR operation can then proceed as normal with the assurance that the distal femoral cuts are in the correct position for attachment of a prosthetic femoral component.

A tibial component 180, according to a further aspect of the present invention, is shown in FIGS. 13 and 14. This is substantially the same as the trial tibial component 102 described earlier in relation to FIG. 5a and like parts are numbered accordingly. The main difference between this tibial component 180 and that described earlier is that each wing 112 has a smooth rather than serrated free edge. Accordingly, the tibial component 180 of FIGS. 13 and 14 is configured as a prosthetic, rather than a trial, tibial component.

As can be seen more clearly in FIGS. 13 and 14, the annular protrusion 116 is disposed towards the edge of the plateau 104 and is triangular in nature. It comprises a substantially vertical inner wall 182 extending away from the plateau to a pointed tip 184 and an inclined outer wall 186 extending from the tip 184 to a point inset from the edge of the plateau 104. As shown in FIG. 14, the portion of the distal surface of the plateau 104 enclosed within the annular protrusion 116 is recessed slightly from that outside the protrusion 116. This allows for a finite thickness of cement mantle when the tibial component 180 is fixed in position.

The tibial component 102 shown in FIGS. 5a through 12 is configured as a trial tibial component. This is generally used during the distraction procedure to determine the correct flexion gap, as described earlier. However, it is replaced by a prosthetic tibial component before being implanted into the tibia. The advantage of this approach is that there is less risk of damage to the prosthetic tibial component than if it were used throughout the entire procedure. That said it would still be possible to carry out the above-described steps on a prosthetic tibial component or even directly on a resected tibia. Preferably, in either of these cases, the tip 56 of the distraction device 62 is sufficiently blunt to minimise contact damage to the prosthetic tibial component/actual tibia.

When configured as a trial tibial component 102 as per FIGS. 5a through 12, the annular protrusion 116 is configured to create an annular cut in the proximal end of a tibia. However, when configured as a prosthetic tibial component 180 as per FIGS. 13 and 14 the annular protrusion 116 is configured to locate in an annular cut, such as that made by the trial tibial component 102 of FIGS. 5a through 12, in the proximal end of a tibia. As shown in FIG. 14, the annular protrusion 116 acts as a cement pressurising flange to aid attachment of the prosthetic tibial component 180 to the proximal end of the tibia.

The various components described above are configured for use during TKR surgery. A summary of the steps that may be employed in a particular operation is as follows:
1 Measure extension gap with leg straight
2 Insert intramedullary rod 10 into femur 100
3 Attach distraction device 62 to rod 10—see FIG. 3a
4 Locate cutting guide 70 over distraction device 62—see FIGS. 4c and 4d
5 Operate distraction device 62 (between rod 10 and trial tibial component 102) to establish the desired collateral ligament tension with the knee flexed at 90°—see FIG. 5b
6 Insert appropriate spacers 120 to obtain a flexion gap equal to the previously measured extension gap—see FIG. 6
7 Allow cutting guide 70 to rest on spacers 120
8 Use anti notch guide 130 to test for positioning of cutting guide 70 relative to anterior of femur 100—see FIG. 8
9 Replace cutting guide 70 with one of different size if necessary
10 Repeat Steps 8 and 9 until anti notch guide 130 just touches femur 100 with cutting guide 70 resting on spacers 120
11 Insert temporary pins 126
12 Use rotational protractor 150/170 to measure rotational position of cutting guide 70 relative to femur 100—see FIG. 10
13 If relative angle between femoral condyles and cutting guide 70 is not in required range, perform soft tissue release and repeat Steps 2 to 13
14 Fix position of cutting guide 70
15 Perform cuts through cutting guide 70
16 Remove instruments
17 Attach femoral component to cut distal end of femur 100.

In the above sequence, the order of Steps 2, 3 and 4 may vary. In addition, Step 4 may be performed after Step 5 or 6.

According to a further aspect of the invention, a kit for use in knee surgery comprises two or more of the following instruments:
a) an intramedullary rod 10 for insertion into an end of a femur 100—see FIG. 1a;
b) a distraction device 62 coupleable to an intramedullary rod 10 and operable between the intramedullary rod 10 and a tibia for adjusting the tension of the collateral ligaments on either side of the knee—see FIG. 3b;
c) a cutting guide 70 configured to locate over an intramedullary rod 10, which has been inserted into an end of a femur 100, such that the position of the cutting guide 70 relative to the intramedullary rod 10, and therefore the femur 100, is adjustable in at least the anterior-posterior direction—see FIG. 5a;
d) an anti-notch guide 130 for providing an indication of the relative positional relationship between a cutting guide 70 and the anterior and/or posterior of the femur 100—see FIG. 7; and
e) a rotation protractor 150/170 configured to measure the degree of rotation between a cutting guide 70 and the femur 100—see FIGS. 9 and 10 or FIGS. 11a and 11b.

It will be appreciated by persons skilled in the art that various modifications may be made to the above-described embodiments without departing from the scope of the present invention. For example, whilst the above discussion has been concerned with TKR, the various aspects of the invention are equally applicable to other types of knee surgery.

1. Instrumentation for use in knee surgery, said instrumentation comprising:
an intramedullary rod for insertion into an end of a femur; and
a distraction device coupleable to the intramedullary rod and operable between the intramedullary rod and a tibia for adjusting the tension of the collateral ligaments on either side of the knee.

2. The instrumentation of claim 1 wherein the distraction device comprises two or more relatively displaceable members.

3. The instrumentation of claim 2 wherein one of said members is configured to be coupled to said intramedullary rod and an other of said members is configured for displacement relative to said one member, in directions which generally correspond to the anterior and posterior directions of the femur when the instrumentation is in use.

4. The instrumentation of claim 3 wherein said one member comprises an internal threaded portion in threaded engagement with a corresponding screw thread provided on said other member.

5. The instrumentation of claim 1 further comprising a device for coupling the distraction device to the tibia.

6. The instrumentation of claim 5 wherein the device is configured to allow adjustment of the position of the distraction device relative to the tibia.

7. The instrumentation of claim 6 wherein the device comprises a socket for receiving a free end of the distraction device, the socket being radially mounted on a wheel configured for rotation in a channel of a trial tibial component, the wheel being rotatably mounted on an axle configured for rolling along a plateau of a trial tibial component.

8. The instrumentation of claim 3 further comprising a tool engageable with said other member and rotatable relative to said one member to facilitate extension and/or contraction of the distraction device.

9. The instrumentation of claim 1 further comprising a cutting guide positionable over said end of the femur such that the position of the cutting guide relative to the femur is adjustable.

10. The instrumentation of claim 9 wherein the cutting guide is configured to locate over the distraction device and the intramedullary rod such as to be positionally adjustable, when the instrumentation is in use, in the anterior-posterior direction relative to the femur.

11. The instrumentation of claim 10 wherein the cutting guide is coupleable to the distraction device and the intramedullary rod by locating the cutting guide over the distraction device and the intramedullary rod through movement in one direction and then sliding the cutting guide relative to the distraction device and the intramedullary rod in an other direction.

12. The instrumentation of claim 10 wherein the cutting guide includes at least one stop to limit the amount of positional adjustability between the cutting guide and the intramedullary rod.

13. The instrumentation of claim 9 further including a guide for providing an indication of the relative positional relationship between the cutting guide and the anterior of the femur.

14. The instrumentation of claim 13 wherein the guide comprises a locating component for engagement with the cutting guide and an indicator spaced apart therefrom such that the indicator is disposed anteriorly of said femur when the instrumentation is in use, thereby to provide an indication of the relative positional relationship.

15. The instrumentation of claim 9 further including a rotation protractor configured to measure the degree of rotation between the cutting guide and the femur.

16. The instrumentation of claim 15 wherein the rotation protractor comprises a reference portion configured to engage said cutting guide when in use and an indicator which extends beneath the lateral and medial condyles of the femur, the indicator being rotatable relative to the reference portion so as to contact the condyles thereby facilitating measurement of the relative angular relationship between the condyles and the cutting guide.

17. Instrumentation for use in knee surgery, said instrumentation comprising:
a cutting guide configured to locate over an intramedullary rod, which has been inserted into an end of a femur, such that the position of the cutting guide relative to the intramedullary rod, and therefore the femur, is adjustable in at least the anterior-posterior direction.

18. A kit for use in knee surgery, said kit comprising two or more of the following instruments:
an intramedullary rod for insertion into an end of a femur;
a distraction device coupleable to an intramedullary rod and operable between the intramedullary rod and a tibia for adjusting the tension of the collateral ligaments on either side of the knee;
a cutting guide configured to locate over an intramedullary rod, which has been inserted into an end of a femur, such that the position of the cutting guide relative to the intramedullary rod, and therefore the femur, is adjustable in at least the anterior-posterior direction;
a guide for providing an indication of the relative positional relationship between a cutting guide and the anterior and/or posterior of the femur; and
a rotation protractor configured to measure the degree of rotation between a cutting guide and the femur.

19. A tibial component for use in knee surgery, said tibial component comprising:
a plateau with a distal surface configured for mating with a proximal end of a resected tibia,
a locating stem extending from said distal surface for insertion into a medullary canal of a said tibia, and
an annular protrusion extending from said distal surface to aid in attachment of the tibial component to a said tibia, when in use.

20. A tibial component of claim 19 configured as a trial tibial component and wherein the protrusion is configured to create an annular cut in a said proximal end of a tibia.

21. A tibial component of claim 20 wherein a recess is provided in the proximal surface of the plateau for receiving a free end of a distraction device, in use.

22. A tibial component of claim 19 configured as a prosthetic tibial component and wherein the protrusion is configured to locate in an annular cut, such as that made by a trial tibial component, in a said proximal end of a tibia.

23. A tibial component of claim 22 wherein the protrusion is configured as a cement pressurising flange to aid attachment of the prosthetic tibial component to a said proximal end of a tibia by increasing the penetration of cement into the cancellous bone of the said proximal end of a tibia when in use.

24. A method of adjusting the tension of the collateral ligaments on either side of a knee, said method comprising the steps of:
inserting an intramedullary rod into an end of a femur; and
operating a distraction device between the intramedullary rod and a tibia to adjust the tension of the collateral ligaments on either side of a said knee.

25. The method of claim 24 further comprising the steps of:
coupling the distraction device to the intramedullary rod prior to the step of operating the distraction device;
coupling the distraction device to a trial tibial component prior to the step of operating the distraction device;
positioning a cutting guide adjacent said end of the femur;
adjusting the relative positioning of the cutting guide with respect to the femur;
inserting a spacer between the tibia and the cutting guide, said spacer being sized to obtain the desired flexion gap;
determining whether the cutting guide extends to the desired position with respect to the anterior of the femur when said cutting guide is allowed to rest on said spacer and, if not, replacing the cutting guide with a different sized cutting guide to achieve the desired position; and
measuring the degree of rotation between the cutting guide and the femur.
